(19) United States
(12) Patent Application Publication
(10) Pub. No.: US 2017/0049916 A1
(43) Pub. Date: Feb. 23, 2017

(54) Sterilizer and Sterilization Method

(71) Applicant: Ikeda Machine Industry Co., Ltd., Osaka-shi (JP)

(72) Inventor: Takeshi Ikeda, Osaka-shi (JP)

(21) Appl. No.: 15/240,217

(22) Filed: Aug. 18, 2016

(30) Foreign Application Priority Data
Aug. 19, 2015 (JP) 2015-161998

(51) Int. Cl. A61L 2/20; B65B 55/18; B65B 55/10

(52) U.S. Cl. CPC A61L 2/20; B65B 55/10; B65B 55/18; A61L 2202/24; A61L 2202/23

(57) ABSTRACT

A sterilizer includes: a first sterilization unit configured to house an object to be sterilized; a second sterilization unit configured to house a bag that houses the object to be sterilized; and a sterilizing gas supply system configured to supply a sterilizing gas to both of the first sterilization unit and the second sterilization unit.

### CROSS-REFERENCE TO RELATED APPLICATION

This application claims priority to Japanese Patent Application No. 2015-161998 filed Aug. 19, 2015, the disclosure of which is hereby incorporated in its entirety by reference.

### BACKGROUND OF THE INVENTION

Field of the Invention

The present invention relates to a sterilizer and a sterilization method in which objects to be sterilized such as endoscopes are sterilized with a sterilizing gas.

Background Art

Conventionally, devices that perform cleaning and sterilization of objects to be sterilized that require sterilization such as endoscopes have been provided. Such a device of this type, for example, includes a cleaning bath for cleaning and disinfecting the objects to be sterilized, and a nozzle for spraying a cleaning liquid.

Then, the objects to be sterilized are set, an antiseptic solution (ozone water) is supplied into the cleaning bath, the objects to be sterilized are immersed in the antiseptic solution, and the cleaning liquid is sprayed to the objects to be sterilized, so that the objects to be sterilized are cleaned and disinfected in the cleaning bath (see JP 2003-135396 A, for example).

Meanwhile, the conventional devices have a problem that it is difficult to reliably sterilize, with ozone water, the outside and inside of the objects to be sterilized on which bacteria are deposited, because the ozone water is used to disinfect the objects to be sterilized.

Further, there are cases where the objects to be sterilized taken out of the device are bagged for storing the cleaned and disinfected objects to be sterilized, but no consideration is given for such bagging in the conventional devices.

### SUMMARY OF THE INVENTION

In view of such an actual situation, it is therefore an object of the present invention to provide a sterilizer and a sterilization method that are capable of reliably sterilizing the outside and inside of an endoscope on which bacteria are deposited.

The following presents a simplified summary of the invention disclosed herein in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

A sterilizer according to the present invention includes a first sterilization unit configured to house an object to be sterilized; a second sterilization unit configured to house a bag that houses the object to be sterilized; and a sterilizing gas supply system configured to supply a sterilizing gas to both of the first sterilization unit and the second sterilization unit.

According to an aspect of the present invention, it is preferable that the sterilizer further include a sterilizing gas exhaust system configured to exhaust the sterilizing gas from each of the first sterilization unit and the second sterilization unit, the sterilizer being configured so that the sterilizing gas is exhausted from the first sterilization unit and the second sterilization unit via the sterilizing gas exhaust system after the sterilizing gas is supplied from the sterilizing gas supply system to the first sterilization unit and the second sterilization unit.

According to another aspect of the present invention, it is preferable that the sterilizer be configured so that a housing operation of the object to be sterilized into the bag is performed from an outside of the sterilizer by the first sterilization unit including a tank having an opening and a cover configured to open and close the opening, the first sterilization unit being configured so that the object to be sterilized is put into the tank through the opening and the object to be sterilized is taken out of the tank through the opening, and the second sterilization unit and the tank being configured to communicate with each other through the opening.

A sterilization method using a sterilizer including a first sterilization unit configured to house an object to be sterilized, a second sterilization unit configured to house a bag that houses the object to be sterilized, and a sterilizing gas supply system configured to supply a sterilizing gas to both of the first sterilization unit and the second sterilization unit, the sterilization method including a sterilization step that includes: sterilizing the object to be sterilized that is housed in the first sterilization unit by supplying the sterilizing gas from the sterilizing gas supply system into the first sterilization unit; and sterilizing the bag that is housed in the second sterilization unit by supplying the sterilizing gas from the sterilizing gas supply system to the second sterilization unit.

According to an aspect of the present invention, it is preferable that the sterilizer further include a sterilizing gas exhaust system configured to exhaust the sterilizing gas from each of the first sterilization unit and the second sterilization unit, the sterilizer being configured so that the sterilizing gas is exhausted from the first sterilization unit and the second sterilization unit via the sterilizing gas exhaust system after the sterilizing gas is supplied from the sterilizing gas supply system to the first sterilization unit and the second sterilization unit, and after the sterilization step, the sterilizing gas inside the first sterilization unit is exhausted to an outside of the sterilizer through the sterilizing gas exhaust system, and the sterilizing gas inside the second sterilization unit is exhausted to the outside of the sterilizer through the sterilizing gas exhaust system.

According to another aspect of the present invention, it is preferable that the sterilization method further include a bagging step of bagging the object to be sterilized into the bag inside the first sterilization unit or the second sterilization unit after the sterilization step.

According to another aspect of the present invention, it is preferable that, in the sterilization step, the first sterilization unit and the second sterilization unit be separated by a partition so as not to be in communication with each other, so that the first sterilization unit and the second sterilization unit independently perform sterilization, and in the bagging step, the partition that partitions between the first sterilization unit and the second sterilization unit so as not to be in communication with each other be opened so that the first sterilization unit and the second sterilization unit communicate with each other, and the bagging is performed in the second sterilization unit.

### BRIEF DESCRIPTION OF THE DRAWINGS

The foregoing and other features of the present invention will become apparent from the following description and drawings of an illustrative embodiment of the invention in which:

FIG. 1 is a schematic configuration diagram showing a sterilizer according to an embodiment of the present invention;

FIG. 2 is a perspective view showing a plurality of nozzles arranged on the peripheral wall of a tank, and a rotating unit of a basket that is rotatably provided inside the tank;

FIG. 3 is a plan view showing a plurality of holders arranged inside the basket shown in FIG. 2, and a plurality of endoscopes placed on the holders;

FIG. 4 is a perspective view showing the plurality of endoscopes with treatment tool insertion openings placed on the plurality of holders shown in FIG. 3;

FIG. 5 is a flow showing an endoscope exterior cleaning step;

FIG. 6 is a flow showing an endoscope interior cleaning step;

FIG. 7 is a flow showing a step of sterilizing the endoscopes within the tank;

FIG. 8 is a flow showing a step of sterilizing bags within a second chamber; and

FIG. 9 is a flow showing a step of bagging the sterilized endoscopes into the sterilized bags.

### DETAILED DESCRIPTION OF THE PREFERRED EMBODIMENT

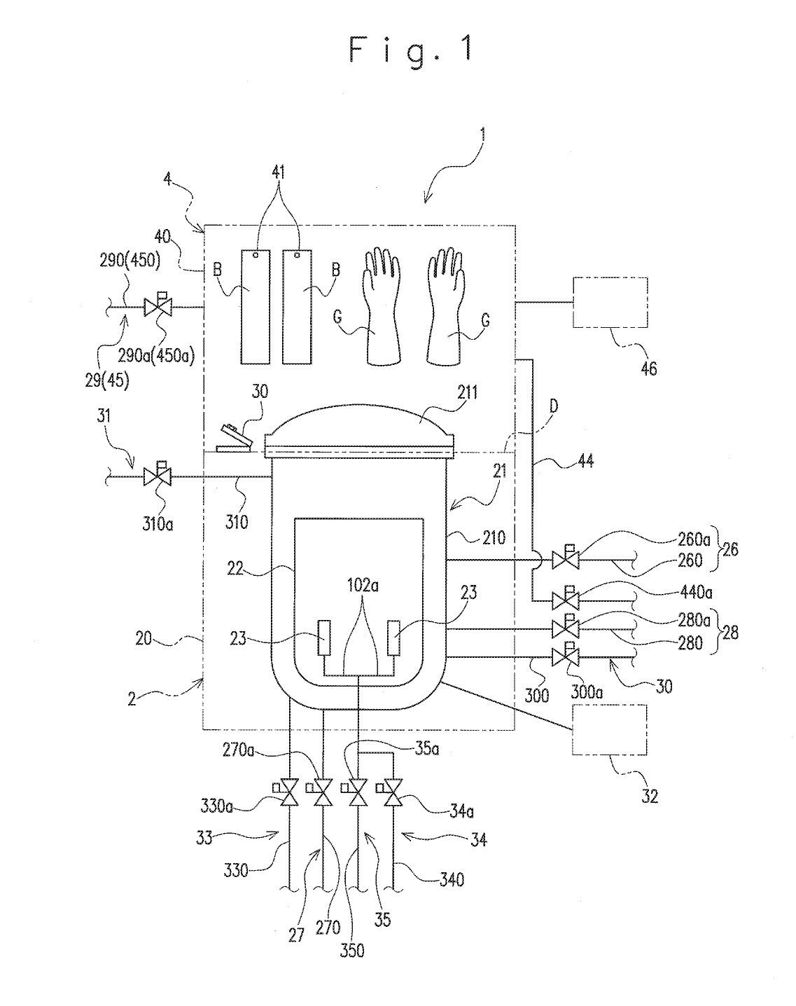

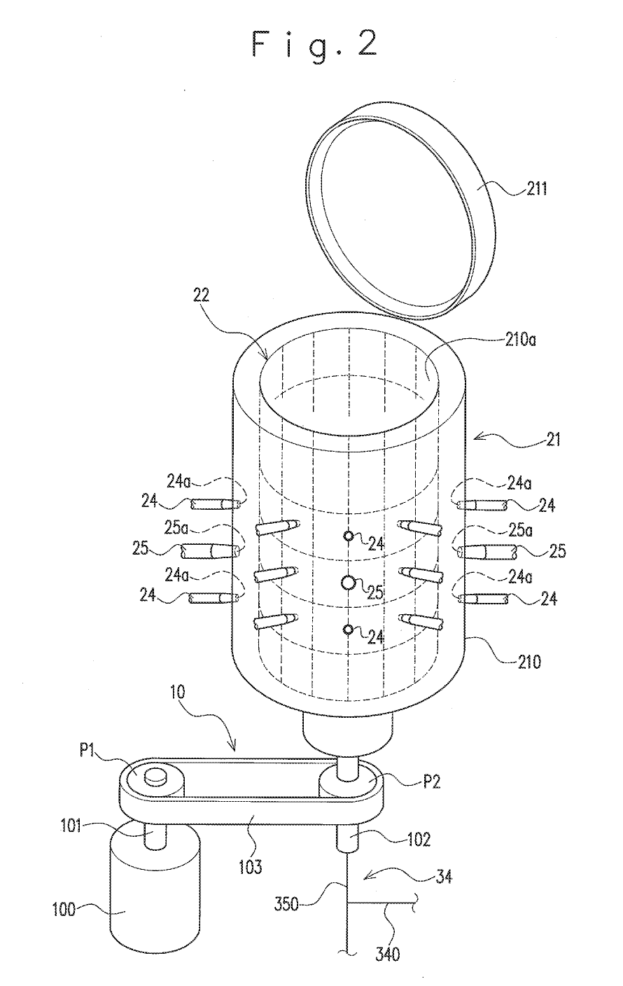

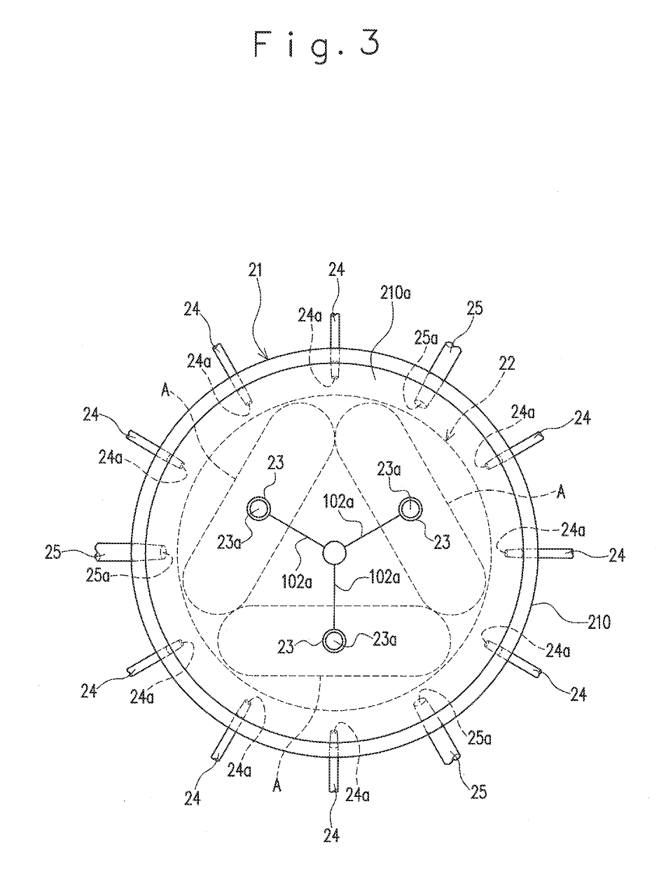

Hereinafter, a sterilizer and a sterilization method according to an embodiment of the present invention will be described with reference to the drawings. As shown in FIG. 1 to FIG. 4, a sterilizer 1 according to this embodiment includes a first sterilization unit 2 capable of housing objects to be sterilized A, a second sterilization unit 4 capable of housing bags B that house the objects to be sterilized A, and a sterilizing gas supply system 28 configured to supply a sterilizing gas to both the first sterilization unit 2 and the second sterilization unit 4. In this embodiment, endoscopes are used as the objects to be sterilized A (hereinafter, referred to as endoscopes A).

As the sterilizing gas, nitrogen dioxide (NO₂), for example, is used in this embodiment.

The first sterilization unit 2 is a housing, and a first chamber 20 is provided in the housing body. A tank 21 for sterilizing the objects to be sterilized A, and a rotating unit 10 configured to rotate a basket 22, which will be described below, relatively to the tank 21 are provided inside the first chamber 20.

The tank 21 includes a tank body 210, a cover 211 configured to open and close an opening 210a of the tank body 210, the basket 22 rotatably provided inside the tank body 210 concentrically with the tank body 210, and holders 23 arranged inside the basket 22 for cleaning the inner sides of the endoscopes A. The cover 211 of the tank body 210 serves as a partition between the inside of the tank body 210 and a second chamber 40. That is, the first sterilization unit 2 and the second sterilization unit 4 are separated by the cover 211 so as not to be in communication with each other, so that they perform sterilization independently, as described below.

The basket 22 is constituted by a cylindrical body having a peripheral wall of 20 mm×20 mm mesh, and has a size such that a plurality of endoscopes A can be housed therein.

The rotating unit 10 includes a motor 100, a pulley P1 fixedly secured to a drive shaft 101 of the motor 100, a rotation shaft 102 extending in the vertical direction from the center of the bottom of the basket 22, a pulley P2 fixedly secured to the rotation shaft 102, and a belt 103 wound around the pulley P1 and the pulley P2.

The rotation shaft 102 is formed into a cylindrical shape. The rotation shaft 102 is configured so that UF water (pure water) can be supplied thereto by high-pressure air, and is branched into a plurality of water supply tubes 102a inside the tank 21, which are connected to base ends (fluid supply passages 23a) of the below described holders 23.

The fluid supply passages 23a for supplying a cleaning liquid and dry air (hot air in this embodiment) to treatment tool insertion openings A1 of the endoscopes A are formed inside the holders 23.

Further, a plurality of nozzles 24 for cleaning the outer sides of the endoscopes A and a plurality of ultrasonic oscillators 25 for cleaning the outer sides of the endoscopes A before the sterilization step are arranged on the peripheral wall of the tank body 210.

The nozzles 24 are arranged on the peripheral wall of the tank body 210 so that ejection ports (tips) 24a face the center of the tank body 210. Specifically, two of the nozzles 24 are each arranged in the axial direction of the tank body 210 at a vertical interval on the peripheral wall of the tank body 210, and further a plurality of sets thereof are arranged throughout the circumferential direction of the tank body 210.

A power supply unit (not shown) configured to supply power for oscillating the plurality of ultrasonic oscillators 25 is provided outside the plurality of ultrasonic oscillators 25. Further, the ultrasonic oscillators 25 are arranged respectively between the sets of two nozzles 24 that are vertically arranged. Specifically, the ultrasonic oscillators 25 are arranged on the peripheral wall of the tank body 210 so that output units (tips) 25a face the center of the tank body 210.

Further, the opening 210a of the tank body 210 has a size such that the endoscopes A can be put in and taken out therethrough. Further, the open end of the tank body 210 is formed through a partition wall D between the first chamber 20 and the second chamber 40. Accordingly, the inside of the tank body 210 and the inside of the second chamber 40 are configured to communicate with each other by opening the cover 211 of the tank body 210.

In the first chamber 20, a cleaning liquid supply system 26, a cleaning liquid drainage system 27, the sterilizing gas supply system 28, a sterilizing gas exhaust system 29, a dry air supply system 30, a dry air exhaust system 31, a humidity controller 32, a vacuum system 33, a cleaning liquid supply system for holders 34 for the holders 23, and a pneumatic system 35 for the holders 23 are provided.

The cleaning liquid supply system 26 supplies a cleaning liquid for cleaning the endoscopes A to the plurality of nozzles 24. Specifically, the cleaning liquid supply system 26 includes a supply tube 260 configured to supply a cleaning liquid to the tank 21 by jet injection, and an on-off valve 260a provided in the supply tube 260.

The cleaning liquid drainage system 27 drains the cleaning liquid in the first chamber 20 supplied from the plurality of nozzles 24 to the outside of the sterilizer 1. Specifically, the cleaning liquid drainage system 27 includes a drainage tube 270 configured to drain, to the outside of the sterilizer 1, pure water supplied to the tank 21, and an on-off valve 270a provided in the drainage tube 270.

In this embodiment, pure water (UF water) is used as the cleaning liquid. Further, the amount of cleaning liquid is appropriately adjusted to be supplied, corresponding to the number and size of the endoscopes A.

The sterilizing gas supply system 28 includes a gas supply tube 28a for supplying the sterilizing gas from the sterile gas supply source (not shown) into the tank 21, and an on-off valve 280a provided in the gas supply tube 28a.

The dry air supply system 30 dries the inside of the tank 21 and the outer sides of the endoscopes A after the cleaning step by dry air (hot air) from the nozzles 24, and dries the inner sides of the endoscopes A by dry air (hot air) from the holders 23. Further, the dry air supply system 30 includes a dry air supply source (not shown) configured to supply air for drying (hot air), a dry air supply tube 300 connected to the dry air supply source and configured to supply dry air (hot air) for drying the tank 21 and the second chamber 40, and an on-off valve 300a provided in the dry air supply tube 300.

The dry air exhaust system 31 includes a dry air exhaust tube 310 configured to exhaust the dry air (hot air) after drying the tank 21 and the second chamber 40 to the outside of the sterilizer 1, and an exhaust valve 310a provided in the dry air exhaust tube 310.

Further, a humidity controller 32 configured to adjust the humidity inside the tank 21 is provided inside the tank 21.

The vacuum system 33 includes a pressure adjusting pipe 330 for evacuating the insides of the tank 21 and the endoscopes A in order to adjust the environment to supply the sterilizing gas, and an on-off valve 330a provided in the pressure adjusting pipe 330.

The cleaning liquid supply system for holders 34 includes a supply tube 340 configured to supply pure water to the fluid supply passages 23a of the holders 23, and an on-off valve 34a provided in the supply tube 340. Further, the pneumatic system 35 includes a high-pressure air pipe 350 configured to supply high-pressure air for cleaning the inner sides of the endoscopes A by jet injection, and a high-pressure air supply valve 35a provided in the high-pressure air pipe 350.

The second chamber 40 is a chamber formed by being surrounded by a plurality of transparent plate materials. The second chamber 40 communicates with the inside of the tank 21 when the cover 211 is opened. The second chamber 40 is configured to allow the operation to fill the bags B with the endoscopes A taken out through the opening 210a of the tank body 210 after the sterilization of the endoscopes A.

Further, in the second chamber 40, bag holding parts 41 holding the plurality of bags B, gloves G for performing the bagging operation from the outside of the second chamber 40, a sealing unit 30 configured to seal the openings of the bags B after the endoscopes A are bagged, a sterilizing gas supply system 44, a sterilizing gas exhaust system 45, and a humidity controller 46 are arranged.

The gloves G are used for performing an operation inside the second chamber 40 by an operator inserting hands from the outside of the sterilizer 1.

The bags B have a size capable of housing the endoscopes A, and are configured to allow the sterilizing gas to pass therethrough from the inside to the outside and from the outside to the inside of the bags B by being made of non-woven fabrics.

The sealing unit 30 is a hand sealer configured to heat-seal the openings of the bags B in this embodiment.

The sterilizing gas supply system 44 includes a gas supply tube 44 for supplying the sterilizing gas from the gas supply source (not shown) into the second chamber 40, and an on-off valve 440a provided in the gas supply tube 44.

The sterilizing gas exhaust system 45 includes a gas exhaust tube 450 (290) configured to exhaust the sterilizing gas from the second chamber 40 to the outside of the sterilizer 1 such as a specific exhaust treatment device (not shown), and an on-off valve 450a (290a) provided in the gas exhaust tube 450 (290). In this embodiment, the sterilizing gas exhaust system 45 of the second chamber 40 serves also as the sterilizing gas exhaust system 29 of the first chamber 20.

Though not shown in the figures, a strainer or filter is appropriately arranged on the drain outlet configured to drain the cleaning liquid accumulated inside the tank 21 after the cleaning.

Further, a concentration sensor for detecting the concentration of the sterilizing gas and a temperature sensor for detecting the temperature of the dry air (hot air) are arranged in each of the inside of the tank 21 and the inside of the second chamber 40.

Next, the sterilization method according to this embodiment will be described. The sterilization method includes: an endoscope exterior cleaning step of cleaning the outer sides of endoscopes A by jet injection of a cleaning liquid from a plurality of nozzles 24; an ultrasonic cleaning step of ultrasonically cleaning the outer sides of the endoscopes A after the endoscope exterior cleaning step; an endoscope interior cleaning step of cleaning the inner sides of the endoscopes A by jet injection of the cleaning liquid from a plurality of holders 23; a sterilization step of sterilizing the outer sides and inner sides of the endoscopes A with a sterilizing gas; and a housing (bagging) step of bagging the sterilized endoscopes A into sterilized bags B after the sterilization step. In this embodiment, jet injection cleaning and ultrasonic cleaning of the endoscopes A are repeated.

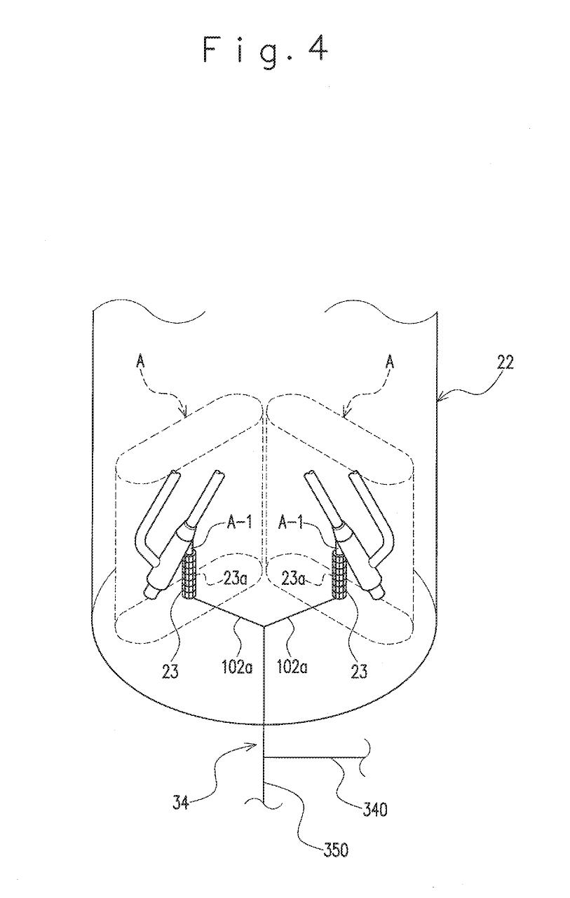

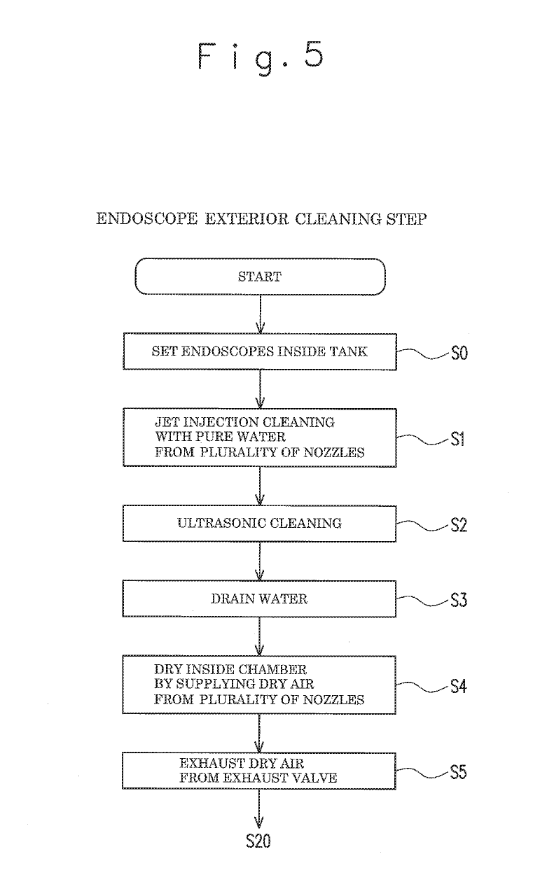

First, in the endoscope exterior cleaning step, the second chamber 40 is opened, the cover 211 of the tank 21 is opened, and the opening 210a of the tank 21 is opened, as shown in FIG. 5. Then, a plurality of endoscopes A (three in this embodiment) are sequentially put into the tank 21 through the opening 210a, and the plurality of endoscopes A are sequentially set at the bottom of the basket 22 inside the tank 21 (step S0). Specifically, as shown in FIG. 4, while winding up the flexible tubes of the endoscopes A, the treatment tool insertion openings A1 of the endoscopes A are placed on the openings of the fluid supply passages 23a of the holders 23.

Next, the cover 211 of the tank 21 is closed so as not to be in communication with the second chamber 40, and the opening 210a of the tank 21 is closed. Then, pure water is supplied while applying a pressure to the plurality of nozzles 24, simultaneously with which the basket 22 is rotated at a specific rotational speed by the rotating unit 10. Thus, the outer sides of the endoscopes A are cleaned by jet injection (step S1).

Next, the process proceeds to the ultrasonic cleaning step (step S2). That is, power source is supplied to the ultrasonic oscillators 25 to cause oscillation, and bacteria deposited on the outer sides of the endoscopes A are removed by ultrasonic vibration. Specifically, ultrasonic vibration generates a numerous number of air bubbles in an ultrasound conductive liquid, and shock waves are caused when the generated air bubbles collide with the endoscopes A to break, so that the bacteria adhering onto the outer sides of the endoscopes A can be removed.

Thereafter, the rotation of the basket 22 is stopped, and the water used in the jet injection cleaning and ultrasonic cleaning is drained to the outside of the sterilizer 1 (step S3).

Next, high-pressure dry air (hot air in this embodiment) is supplied to the plurality of nozzles 24 through the dry air supply tube 300 and the on-off valve 300a, and the inside of the tank 21 is dried by the dry air supplied from the plurality of nozzles 24 (step S4). After drying, the dry air is exhausted to the outside of the sterilizer 1 through the dry air exhaust tube 310 and the exhaust valve 310a (step S5). This can prevent occurrence of bacteria due to the moisture adhering onto the outer sides of the endoscopes A.

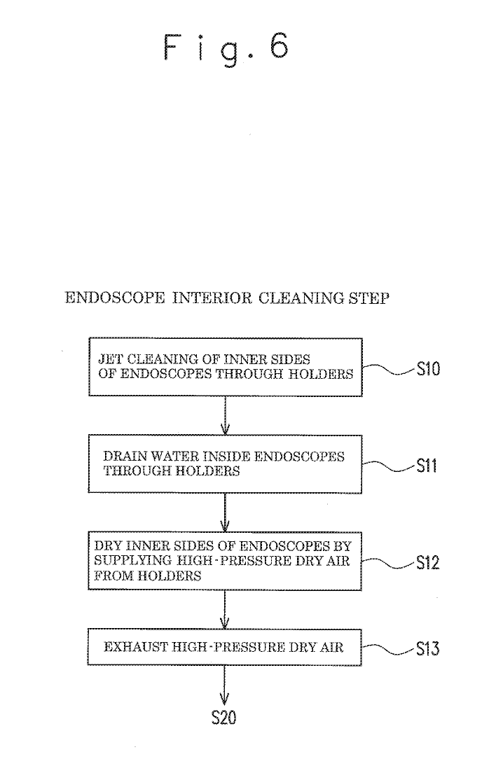

Meanwhile, the endoscope interior cleaning step is performed concurrently with the cleaning of the outer sides of the endoscopes A, as shown in FIG. 6. That is, the inner sides of the endoscopes A are cleaned by jet injection (step S10). Specifically, pure water is supplied to the fluid supply passages 23a of the holders 23 at high pressure, and the inner sides of the endoscopes A are cleaned. Thereafter, the water inside the endoscopes A is drained through the holders 23 (step S11). In this embodiment, jet injection cleaning and ultrasonic cleaning of the endoscopes A are repeated.

Next, high-pressure dry air (hot air in this embodiment) is supplied into the endoscopes A through the fluid supply passages 23a of the holders 23, and the inner sides of the endoscopes A are dried (step S12). After the drying, the dry air is exhausted to the outside of the sterilizer 1 (step S13). This can prevent occurrence of bacteria due to the moisture remaining on the inner sides of the endoscopes A.

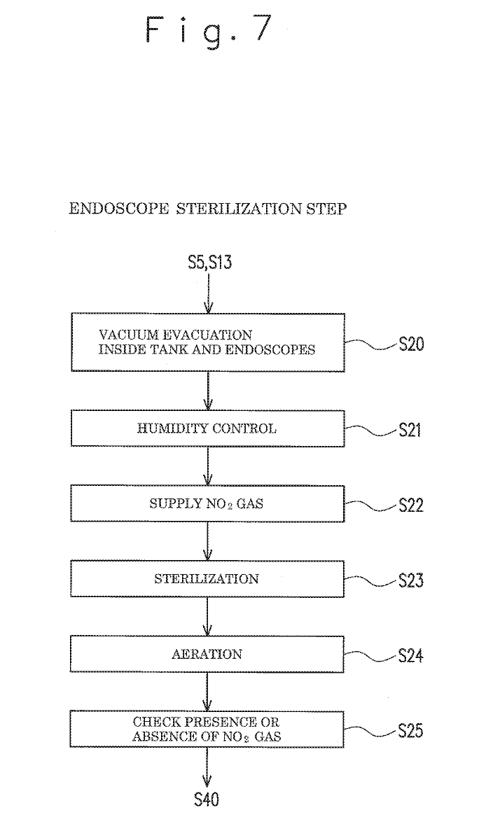

Next, the process proceeds to the sterilization step of the endoscopes A. First, as shown in FIG. 7, the inside of the tank 21 and the insides of the endoscopes A are evacuated through the vacuum tube 330 and the on-off valve 330a (step S20). Thereafter, the humidity controller 32 adjusts the humidity inside the tank 21 (including the insides of the endoscopes A) (step S21). That is, an environment in which the sterilizing gas can be supplied to the second chamber 40 and the inside of the tank 21 (including the insides of the endoscopes A) is adjusted.

Then, a specific amount of sterilizing gas is supplied from the sterile gas supply source (step S22), and the endoscopes A and the inside of the tank 21 of the first chamber 20 are sterilized with the specific amount of sterilizing gas supplied (step S23).

Then, the inside of the tank 21 (including the insides of the endoscopes A) is aerated (step S24). Specifically, the aeration of the inside of the tank 21 (including the insides of the endoscopes A) is performed by supplying clean air multiple times. Here, in the case where the sterilizing gas is discharged from the inside of the tank 21 in a short time, supply of the clean air and vacuum evacuation of the gas inside the tank 21 may be repeated. This reduces the concentration of the sterilizing gas inside the tank 21 (including the insides of the endoscopes A).

Next, the presence or absence of sterilizing gas remaining inside the tank 21 (including the insides of the endoscopes A) is checked using the concentration detection sensor (step S25).

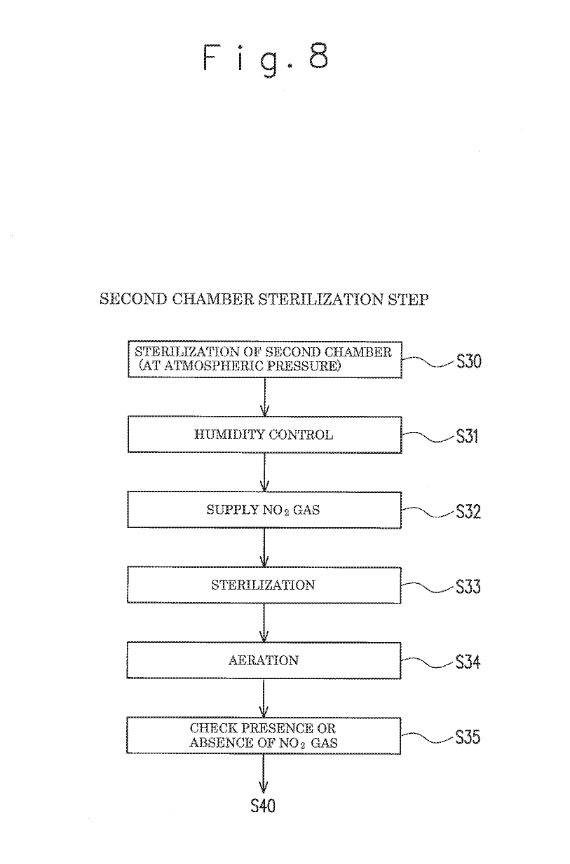

Meanwhile, as shown in FIG. 8, after the cover 211 in the tank 21 is closed so that the second chamber 40 and the tank 21 do not communicate with each other, the process proceeds to the sterilization step of the second chamber 40 at atmospheric pressure (step S30). In the sterilization step, after the humidity inside the second chamber 40 is adjusted by the humidity controller 46 (step S31), the sterilizing gas is supplied (step S32). Then, a plurality of the bags B in the second chamber 40 are sterilized (step S33).

Thereafter, the second chamber 40 is aerated (step S34). Specifically, the cover 211 of the tank 21 is opened, and the gas inside the second chamber 40 and the tank 21 is exhausted to the outside of the sterilizer 1 while clean air is supplied thereto. This reduces the concentration of the sterilizing gas inside the second chamber 40.

Next, the presence or absence of sterilizing gas remaining inside the second chamber 40 is checked using the concentration detection sensor (step S35).

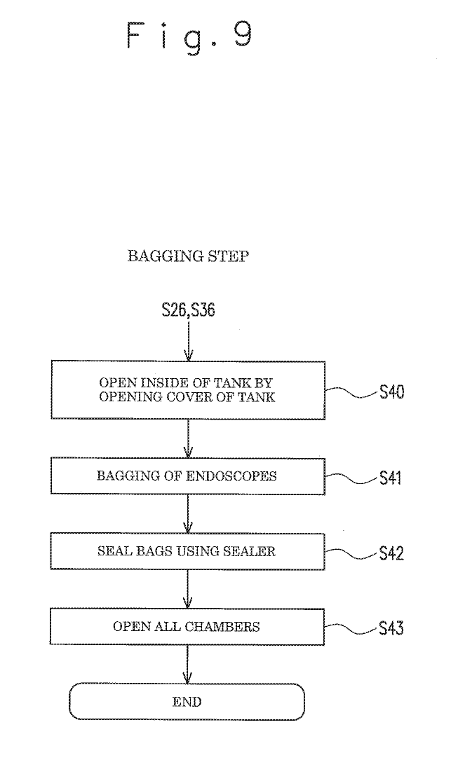

After the sterilization step, as shown in FIG. 9, the process proceeds to the bagging step. Specifically, the cover 211 of the tank 21 is opened, and the opening 210a of the tank 21 is opened (step S40). Then, the endoscopes A are taken out through the opening 210a of the tank 21 to be bagged into the bags B (step S41), and the openings of the bags B are sealed using the hand sealer (step S42).

Then, the second chamber 40 and the cover 211 of the tank 21 are opened (step S43), and the plurality of endoscopes A thus bagged are conveyed to the outside.

Finally, the configuration and action of the aforementioned embodiments (including modifications) will be summarized. The sterilizer 1 according to the aforementioned embodiments includes the first sterilization unit 2 configured to house the objects to be sterilized A; the second sterilization unit 4 configured to house the bags B that house the objects to be sterilized A; and the sterilizing gas supply system 28 configured to supply the sterilizing gas to both of the first sterilization unit 2 and the second sterilization unit 4.

According to such a configuration, the objects to be sterilized A can be directly sterilized with the sterilizing gas in the first sterilization unit 2, and the bags B can be directly sterilized with the sterilizing gas in the second sterilization unit 4. Therefore, the sterilization effect is high, and the objects to be sterilized A can be reliably sterilized.

It is preferable that the sterilizer 1 further include the sterilizing gas exhaust system 29 configured to exhaust the sterilizing gas from each of the first sterilization unit 2 and the second sterilization unit 4, so that the sterilizing gas is exhausted from the first sterilization unit 2 and the second sterilization unit 4 via the sterilizing gas exhaust system 29, after the sterilizing gas is supplied from the sterilizing gas supply system 28 to the first sterilization unit 2 and the second sterilization unit 4.

According to the aforementioned example of the configuration, the sterilization can be reliably performed by supplying the sterilizing gas to each of the first sterilization unit 2 and the second sterilization unit 4, and the exhaust can be performed in a short time without the sterilizing gas remaining in the first sterilization unit 2 and the second sterilization unit 4 by exhausting the sterilizing gas from the first sterilization unit 2 and the second sterilization unit 4.

It is preferable that the sterilizer 1 be configured so that the housing operation of the objects to be sterilized A into the bags B is performed from the outside of the sterilizer 1 by the first sterilization unit 2 including the tank 21 having the opening 210a and the cover 211 configured to open and close the opening 210a, the first sterilization unit 2 being configured so that the objects to be sterilized A are put into the tank 21 through the opening 210a and the objects to be sterilized A are taken out of the tank 21 through the opening 210a, and the second sterilization unit 4 and the tank 21 being configured to communicate with each other through the opening 210a.

According to the aforementioned example of the configuration, the objects to be sterilized A can be housed in the bags B inside the sterilized second sterilization unit 4 or inside the sterilized tank 21. Therefore, contamination of the objects to be sterilized A and the insides of the bags B after the sterilization can be suppressed.

The sterilization method according to the aforementioned embodiments using the sterilizer 1 including the first sterilization unit 2 configured to house the objects to be sterilized A, the second sterilization unit 4 configured to house the bags B that house the objects to be sterilized A, and the sterilizing gas supply system 28 configured to supply the sterilizing gas to both of the first sterilization unit 2 and the second sterilization unit 4, the sterilization method including the sterilization step that includes: sterilizing the objects to be sterilized A that are housed in the first sterilization unit 2 by supplying the sterilizing gas from the sterilizing gas supply system 28 into the first sterilization unit 2; and sterilizing the bags B housed in the second sterilization unit 4 by supplying the sterilizing gas from the sterilizing gas supply system 28 to the second sterilization unit 4.

According to the aforementioned example of the configuration, the endoscopes A as the objects to be sterilized A and the bags B housing the endoscopes A are individually sterilized with the sterilizing gas. Therefore, the endoscopes A and the bags B housing the endoscopes A can be reliably brought into sterile condition.

It is preferable that the sterilizer 1 further include the sterilizing gas exhaust system 29 configured to exhaust the sterilizing gas from each of the first sterilization unit 2 and the second sterilization unit 4, the sterilizer 1 being configured so that the sterilizing gas is exhausted from the first sterilization unit 2 and the second sterilization unit 4 via the sterilizing gas exhaust system 29 after the sterilizing gas is supplied from the sterilizing gas supply system 28 to the first sterilization unit 2 and the second sterilization unit 4, and after the sterilization step, the sterilizing gas inside the first sterilization unit 2 is exhausted to the outside through the sterilizing gas exhaust system 29, and the sterilizing gas inside the second sterilization unit 4 is exhausted to the outside through the sterilizing gas exhaust system 29.

According to the aforementioned example of the configuration, the sterilization can be reliably performed by supplying the sterilizing gas to each of the first sterilization unit 2 and the second sterilization unit 4, and the exhaust can be performed in a short time without the sterilizing gas remaining in the first sterilization unit 2 and the second sterilization unit 4 by exhausting the sterilizing gas from the first sterilization unit 2 and the second sterilization unit 4.

It is preferable that the sterilization method further include the bagging step of bagging the objects to be sterilized A into the bags B inside the first sterilization unit 2 or the second sterilization unit 4 after the sterilization step.

According to the aforementioned example of the configuration, the bagging can be performed under sterile environment by performing the bagging inside the first sterilization unit 2 or the second sterilization unit 4. Therefore, the objects to be sterilized A can be reliably sterilized.

It is preferable that, in the sterilization step, the first sterilization unit 2 and the second sterilization unit 4 be separated by a partition so as not to be in communication with each other so that the first sterilization unit 2 and the second sterilization unit 4 independently perform sterilization, and in the bagging step, the partition that partitions between the first sterilization unit 2 and the second sterilization unit 4 so as not to be communication with each other be opened so that the first sterilization unit and the second sterilization unit communicate with each other, and the bagging is performed in the second sterilization unit.

According to the aforementioned example of the configuration, the objects to be sterilized A and the bags B can be reliably sterilized respectively inside the first sterilization unit 2 and the second sterilization unit 4 that are not in communication with each other. Thereafter, the first sterilization unit 2 and the second sterilization unit 4 are brought into communication with each other, and the bagging operation is performed inside the second sterilization unit 4. Therefore, the sterilization method with the aforementioned configuration allows the bagging operation to be performed while maintaining the sterile condition.

According to the aforementioned example of the configuration, this embodiment can exert an excellent effect that the bacteria deposited on the outer sides and inner sides of the endoscopes A as the objects to be sterilized A can be reliably sterilized to be brought into sterile condition.

It is a matter of course that the present invention is not limited to the above described embodiments, and various modifications can be made without departing from the scope of the invention of the present invention.

For example, in the aforementioned embodiments, jet injection is performed from the plurality of nozzles 24 to the endoscopes A by rotating the basket 22, but jet injection may be performed from the plurality of nozzles 24 to the endoscopes by rotating the tank 21 or by rotating both of the tank 21 and the basket 22.

Further, in the aforementioned embodiments, the second chamber 40 is arranged on the first chamber 20 in a vertical column, but the first chamber 20 and the second chamber 40 may be arranged in a horizontal row.

Further, in the aforementioned embodiments, the bags into which the endoscopes A are bagged are made of non-woven fabrics, but there is no limitation to this. The material needs only to be permeable to the sterilizing gas and allow sterilization of the inner sides and outer sides of the bags B.

Further, in the aforementioned embodiments, the specific amount of sterilizing gas is supplied into the tank 21 and the second chamber 40. However, sensors that detect the concentration of the sterilizing gas may be attached respectively to the inside of the tank 21 and the second chamber 40, and the sterilizing gas may be supplied until the concentration of the sterilizing gas inside the tank 21 and the second chamber 40 reaches a specific value.

Further, in the aforementioned embodiments, the second chamber 40 is at atmospheric pressure when the inside of the tank 21 is evacuated, but the inside of the tank 21 and the second chamber 40 may be evacuated.

Further, in the aforementioned embodiments, the three endoscopes A are set in the single basket 22, but the capacity of the single basket 22 may be increased so that the number of the endoscopes A to be set is increased, or a plurality of endoscopes A may be set respectively in a plurality of baskets 22, thereby improving the operational efficiency in sterilizing the endoscopes A.

Further, in the aforementioned embodiments, the sterilizing gas exhaust system 45 of the second chamber 40 serves also as the sterilizing gas exhaust system 29 of the first chamber 20, but they may be individually provided.

The sterilizer and the sterilization method of the present embodiments are as described above. However, the present invention is not limited to the above described embodiments, and the design can be appropriately modified within the scope intended by the present invention. The operational advantage of the present invention is also not limited to the foregoing embodiments. The embodiments disclosed herein should be construed in all respects as illustrative but not limiting. The scope of the present invention is not indicated by the foregoing description but by the scope of the claims. Further, the scope of the present invention is intended to include all the modifications equivalent in the sense and the scope to the scope of the claims

What is claimed is:

1. A sterilizer comprising:
a first sterilization unit configured to house an object to be sterilized;
a second sterilization unit configured to house a bag that houses the object to be sterilized; and
a sterilizing gas supply system configured to supply a sterilizing gas to both of the first sterilization unit and the second sterilization unit.

2. The sterilizer according to claim 1, further comprising:
a sterilizing gas exhaust system configured to exhaust the sterilizing gas from each of the first sterilization unit and the second sterilization unit, the sterilizer being configured so that the sterilizing gas is exhausted from the first sterilization unit and the second sterilization unit via the sterilizing gas exhaust system after the sterilizing gas is supplied from the sterilizing gas supply system to the first sterilization unit and the second sterilization unit.

3. The sterilizer according to claim 1, wherein the sterilizer is configured so that a housing operation of the object to be sterilized into the bag is performed from an outside of the sterilizer by the first sterilization unit comprising a tank having an opening and a cover configured to open and close the opening, the first sterilization unit being configured so that the object to be sterilized is put into the tank through the opening and the object to be sterilized is taken out of the tank through the opening, and the second sterilization unit and the tank being configured to communicate with each other through the opening.

4. A sterilization method using a sterilizer comprising a first sterilization unit configured to house an object to be sterilized, a second sterilization unit configured to house a bag that houses the object to be sterilized, and a sterilizing gas supply system configured to supply a sterilizing gas to both of the first sterilization unit and the second sterilization unit, the sterilization method comprising a sterilization step that comprises:
sterilizing the object to be sterilized that is housed in the first sterilization unit by supplying the sterilizing gas from the sterilizing gas supply system into the first sterilization unit; and
sterilizing the bag that is housed in the second sterilization unit by supplying the sterilizing gas from the sterilizing gas supply system to the second sterilization unit.

5. The sterilization method according to claim 4, wherein
the sterilizer further comprises a sterilizing gas exhaust system configured to exhaust the sterilizing gas from each of the first sterilization unit and the second sterilization unit, the sterilizer being configured so that the sterilizing gas is exhausted from the first sterilization unit and the second sterilization unit via the sterilizing gas exhaust system after the sterilizing gas is supplied from the sterilizing gas supply system to the first sterilization unit and the second sterilization unit, and
after the sterilization step, the sterilizing gas inside the first sterilization unit is exhausted to an outside of the sterilizer through the sterilizing gas exhaust system, and the sterilizing gas inside the second sterilization unit is exhausted to the outside of the sterilizer through the sterilizing gas exhaust system.

6. The sterilization method according to claim 4, further comprising:
a bagging step of bagging the object to be sterilized into the bag inside the first sterilization unit or the second sterilization unit after the sterilization step.

7. The sterilization method according to claim 6 wherein
in the sterilization step, the first sterilization unit and the second sterilization unit are separated by a partition so as not to be in communication with each other, so that the first sterilization unit and the second sterilization unit independently perform sterilization, and
in the bagging step, the partition that partitions between the first sterilization unit and the second sterilization unit so as not to be in communication with each other is opened so that the first sterilization unit and the second sterilization unit communicate with each other, and the bagging is performed in the second sterilization unit.
